(19) United States
(12) Patent Application Publication
(10) Pub. No.: US 2008/0102137 A1
(43) Pub. Date: May 1, 2008

(54) Composition and method for etiological treatment and prevention of diseases and/or complications associated with chronic glucose metabolism destabilization

(76) Inventor: Manning V.R. Guffey, McAllen, TX (US)

Correspondence Address: P. JEFF MARTIN;THE McGOUGAN LAW FIRM, LLC, 2120 SEA MOUNTAIN HWY., SUITE 200, P.O. BOX 4369, N. MYRTLE BEACH, SC, 29597, US

(21) Appl. No.: 11/590,264

(22) Filed: Oct. 31, 2006

(51) Int. Cl. A61K 33/26; A61K 31/714; A61K 31/455; A61K 31/525; A61K 31/385; A61K 31/355; A61K 33/06

(52) U.S. Cl. 424/641; 514/52; 514/251; 514/250; 514/168; 514/350; 514/276; 514/356; 514/440; 514/393; 514/561; 514/562; 514/565; 424/725; 424/728; 424/757; 424/773; 424/768; 514/474; 514/458; 514/690; 424/195.17; 424/646; 424/702; 424/682

(57) ABSTRACT

A natural composition formulated to modulate multiple pathophysiological processes in order to facilitate homeostatic glucose metabolism in a patient, and more particularly to increase insulin sensitivity in the same. The natural composition is a safe and effective formulation for the etiological prevention and treatment of diseases, complications, conditions, or disorders associated with chronic or long-term destabilization of glucose metabolism.

### RELATED APPLICATIONS

The present invention was first described in Disclosure Document No. 605,321 filed on Aug. 30, 2006 under 35 U.S.C. §122, 37 C.F.R. §1.14, and MPEP §1706. It is respectfully requested that said Disclosure Document remain a permanent part of the file history of the present application and be relied upon during the pending prosecution, and for any other matters that may arise concerning said present application and the subject matter contained therein. There are no previously filed, nor currently any co-pending applications, anywhere in the world.

### BACKGROUND OF THE INVENTION

1. Field of the Invention

The present invention relates generally to methods and compositions for increasing insulin sensitivity and, more particularly, to a method and composition for the etiological prevention and treatment of diseases, complications, conditions, and/or disorders associated with chronic or long-term destabilization of glucose metabolism.

2. Description of the Related Art

Glucose homeostasis is accomplished through intricate, and arguably, elegant interactions among several organs and hormones. Glucose homeostasis depends upon the balance between hepatic glucose production and glucose utilization by insulin-dependent tissues, such as muscle, adipose cells, and liver, and further by insulin-independent tissues such as kidney and brain.

This balance is controlled by pancreatic endocrine hormones, insulin from the β-cell of the pancreatic islet and glucagon from the α-cell. Insulin and glucagon serve as acute regulators of blood glucose concentration.

Insulin dependent diabetes mellitus (IDDM) or Type I diabetes mellitus is characterized by a progressive loss of insulin secretory function by β-cells of the pancreatic islet. Type I diabetes mellitus is characterized by the following clinical signs or symptoms: polydipsia and/or hyperphagia; persistently elevated plasma glucose concentration or hyperglycemia; polyuria; chronic microvascular complications such as retinopathy, nephropathy and neuropathy; and macrovascular complications such as hyperlipidemia and hypertension which can lead to blindness, end-stage renal disease, limb amputation and myocardial infarction (heart attack).

Non-insulin dependent mellitus (NIDDM), also known as Type II diabetes mellitus or adult-onset diabetes mellitus, is a highly prevalent metabolic disorder of glucose metabolism. Type II diabetes comprises approximately 90% of all diabetes cases. The onset of this disease usually occurs in humans at about middle age, however, signs of the conditions may be discoverable earlier. Alarmingly, Type II diabetes has reached epidemic proportions worldwide. In the U.S. alone, over 16 million people are afflicted with Type II diabetes and approximately 13 million have impaired glucose tolerance. In the next twenty years, it is estimated that approximately 300 million people across the world will have diabetes.

Normal human homeostatic glucose metabolism depends upon three factors: 1) the ability of the pancreas to secrete insulin in response to blood glucose levels; 2) the ability of insulin to induce insulin sensitivity or glucose uptake in peripheral tissues; and 3) the ability of insulin to suppress glucose production in the liver. NIDDM or Type II diabetes is the result of metabolic defects in the first two of these three factors. Type II diabetes is characterized by a problematic decrease in insulin sensitivity, known as insulin resistance, compounded by the inability of the pancreas cells to produce enough insulin to remedy this decreased sensitivity. Chronic hyperglycemia is the trademark result of these dysfunctions.

Type II diabetes mellitus usually develops in adulthood but can advance the aging process. Type II diabetes mellitus is associated with the following clinical signs or symptoms: polyuria; polydipsia and/or hyperphagia; persistently elevated plasma glucose concentration or hyperglycemia; chronic microvascular complications such as retinopathy, nephropathy and neuropathy; and macrovascular complications such as hyperlipidemia and hypertension which can lead to blindness, end-stage renal disease, limb amputation, myocardial infarction, stroke, and peripheral vascular disease.

Insulin Resistance Syndrome or Syndrome X is realized in approximately 2% of diagnostic coronary catheterizations. Insulin Resistance Syndrome presents symptoms for the development of Type II diabetes mellitus and cardiovascular disease, including insulin resistance, hyperinsulinemia, dyslipidemia, impaired glucose tolerance, impaired fasting glucose, hypertension and obesity.

Therapy for patients suffering with Type I diabetes mellitus includes the administration of exogenous insulin. However, the utilization of heterologous species material facilitates formation of anti-insulin antibodies which have activity-limiting effects and result in progressive requirements for larger doses in order to achieve desired hypoglycemic effects.

Therapy for patients suffering with Type II diabetes mellitus includes the maintenance of blood glucose at normal levels coupled with proper diet and exercise. Therapy further includes antidiabetic agent treatment, insulin, or combinations thereof.

First-line therapies generally include thiazolidinediones or glitazones, metformin, and sufonylureas. Thiazonlidinediones or glitazones are the first line of medication designed to reverse the basic problem in Type II diabetes of resistance to insulin. The drugs currently available in this group reverse insulin resistance by improving the sensitivity insulin receptors in muscle, liver, and fat cells. This helps the body use insulin more efficiently. These drugs improve sensitivity partly by reducing levels of inflammatory cytokines, i.e., tumor necrosis factor alpha, while increasing activity of the PPAR gamma receptor. They further prevent the liver from overproducing glucose, and lower blood sugar levels about 15% while simultaneously lowering insulin levels by 20%. In addition to improving insulin sensitivity, thiazolidinediones may decrease cardiac risks. Thiazolidinediones raise low-density lipoprotein (LDL) levels slightly, but increase the size of the LDL molecule. Thiazolidinediones also lower alpha tumor necrosis factor. Triglyceride and blood pressure levels are reduced, while high-density lipoprotein (HDL) levels are slightly raised. However, thiazolidinediones or glitazones may produce side effects which include water retention and ankle swelling, weight gain, muscle weakness, and fatigue.

Metformin is a drug from the biguanide class. Metformin lowers fasting blood glucose levels by an average of 25%, and postprandial blood glucose up to 44.5%. Metformin reduces raised plasma insulin levels in cases of metabolic syndrome by as much as 30% and reduces the need for injected insulin in Type II diabetics by 15% to 32%. Meformin shuts off the liver's excess production of glucose, thus it reduces the amount of injected insulin needed to control the blood sugar in both Type I and Type II diabetes. However, metformin may produce side effects which include lactic acidosis, loss of appetite, change of taste, nausea or vomiting, abdominal bloating or gas, diarrhea, or skin rash.

Sulfonylureas stimulate the β-cells to produce more insulin. Sulfonylureas have eliminated the need for many Type II diabetics to inject insulin. However, sulfonylureas are ineffective for those with Type I diabetes or anyone with Type II diabetes whose β-cells no longer produce insulin. Sulfonylureas may produce side effects which include low blood sugar, bloating, nausea, heartburn, anemia, weight gain, metallic or change in taste.

Chronic or long-term destabilization of glucose metabolism and impaired insulin sensitivity has been shown to be associated or correlated with diseases, complications, or disorders including chronic viral infections, chronic viral disorders, psychological conditions, and conditions representing a homeostatic imbalance.

Diseases associated with glucose homeostatic imbalance include cancer and acquired immunodeficiency syndrome (AIDS). Chronic viral infections associated with glucose homeostatic imbalance include human immunodeficiency virus (HIV) hepatitis, Human Papilloma virus (HPV), Coxsackie B virus, Hauta virus, and herpes such as herpes simplex virus, human herpes 6 virus, Epstein-Barr virus.

HPV is a large family of viruses that infect the skin, often causing irregular cell growth or warts. There are more than 60 types of HPV. Some types of the virus are transmitted by nonsexual personal contact and cause common skin warts. Several types of HPV are spread by sex and primarily infect the genitals or anal area. Of the sexually transmitted types, some cause cancer and pre-cancerous changes of the cervix, anus, or the skin of the penis or female genitals. Other types cause warts of the genitals or anal area. Genital warts are the most common symptoms of HPV infection.

Coxsackie B viruses are members of the Picornaviridae family, genus Enteroviurs. The picorna family is marked by its extremely small size. The virion is a naked icosahedron, about 30 nm diameter. The genome is comprised of single-stranded, monopartite RNA. Enteroviruses persist in the 'stomach and are remarkably resistant to its harsh conditions. Coxsackie B virus diseases can range from relatively minor gastrointestinal upsets to paralysis, cardiac damage, and birth defects, although the subclinical and mild infections are by far the most common. As with most infections, the effects are compounded in developing countries and poor areas where malnutrition and poor access to health care predominate. Enteroviruses generally gain access to the body through the mouth, with fecal-oral being the major mode of transmission although the virus can also be found in the respiratory secretions. Warm weather favors transmission by increasing human contacts and the dissemination of the virus through water. All coxsackie B viruses are associated with a host of numerous and varied syndromes, including meningitis, pericarditis, myocarditis, upper respiratory illness and pneumonia, rash, and hepatitis, to name a few. While hand, foot, and mouth disease is usually caused by coxsackievirus A16, B2 and B5 have also been implicated. Coxsackie B viruses have been increasingly recognized as a cause of myocardial disease in adults and children, with up to 39% of people infected with coxsackievirus B5 developing cardiac abnormalities.

Chronic viral disorders associated with glucose homeostatic imbalance, and particularly insulin resistance, include AIDS.

Psychological conditions associated with glucose homeostatic imbalance include attention deficit disorder (ADD), attention deficit hyperactivity disorder (ADHD) depression, mood swings, anger, anxiety, and confusion.

Conditions representing a homeostatic imbalance associated with glucose metabolism destabilization include chronic fatigue syndrome, inflammation, joint and muscle pain, headaches, and nasal and sinus congestion.

Treatment or therapy regarding the aforementioned diseases, complications, and disorders resulting from chronic destabilization of glucose metabolism and insulin resistance has demonstrated at most, limited to relative success. Treatment has consisted primarily of pharmacological or drug treatment resulting in harmful side effects and exorbitant costs exceeding $100 billion annually. Particularly, insulin resistance with it's associated complications and disorders such as hypertension and hyperlipidemia adds to these melancholic statistics.

Accordingly, a need has arisen for a method and an effective, safe, and natural composition for both the etiological treatment and prevention of diseases, complications, conditions, or disorders associated with chronic or long-term destabilization of glucose metabolism, and particularly insulin resistance in a manner which is quick, easy, and efficient.

A search of the prior art did not disclose any patents that read directly on the claims of the instant invention; however, the following references were considered related.

U.S. Pat. No. 6,258,848 B1, issued in the name of Fanus discloses a method of increasing insulin sensitivity in a subject by administering an effective amount of N-acetyl cysteine.

U.S. Pat. No. 7,084,124 B2, issued in the name of Patel et al. discloses substituted indazole-O-glucosides, compositions containing them, and methods of using them, for example for the treatment of diabetes and Syndrome X.

U.S. Pat. No. 5,723,476, issued in the name of Larsen et al. discloses 4-hydroxycoumarin-3-carboxamide derivatives and the use of a broad class of 4-hydroxycoumarin-3-carboxamide derivatives for the treatment of a patient suffering from or susceptible to diabetes mellitus.

U.S. Pat. No. 7,060,295 B2, issued in the name of Richardson et al. discloses compositions and dosage forms being clinically useful as methods for increasing the effectiveness, efficiency and safety of biguanides (metformin) and/or sulfonylureas in the prevention and treatment of insulin resistance and diabetes mellitus, alone or in combination, as a nutrient for humans.

U.S. Pat. No. 6,485,480 B1, issued in the name of Brewiff discloses treatment methods using homeopathic preparations of growth factors.

U.S. Pat. No. 6,147,098, issued in the name of Mogensen et al. discloses methods, compositions, and uses for substituted guanidines and diaminonitroethenes.

U.S. Pat. No. 6,893,829 B2, issued in the name of Nadler et al. discloses the administration of a human leukocyte 12-lipoxygenase pathway inhibitor to inhibit disease etiology, to inhibit the proliferation of breast cancer and to increase insulin receptor phosphorylation in a patient having Type II diabetics.

Consequently, a need has been felt for a method and an effective, safe, and natural composition adaptively formulated to provide an etiological treatment and etiological prevention for diseases, complications, conditions, or disorders associated with chronic or long-term destabilization of glucose metabolism, and particularly insulin resistance in a manner which is quick, easy, and efficient.

### SUMMARY OF THE INVENTION

Therefore, it is an object of the present invention to provide a natural composition that is adaptively formulated to modulate multiple pathophysiological processes in order to facilitate homeostatic glucose metabolism.

It is another object of the present invention to provide an etiological treatment and prevention for diseases, complications, conditions, or disorders associated with chronic or long-term destabilization of glucose metabolism.

It is another object of the present invention to provide a natural composition adapted to increase insulin sensitivity.

It is another object of the present invention to provide a natural composition that is safe and effective.

It is another object of the present invention to provide a natural composition adapted to be administered in solid or liquid form.

It is another object of the present invention to provide a natural composition comprised of vitamins, fatty acids, amino acids, phytochemicals, trace minerals, antioxidants, and metals.

It is another object of the present invention to provide a natural composition formulated to safely and effectively treat and prevent diseases, conditions, complications, or disorders including chronic viral infections, chronic viral disorders, psychological conditions such as depression, mood swings, anger, anxiety, and confusion, chronic fatigue syndrome, inflammation, joint and muscle pain, headaches, nasal and sinus congestion, attention deficit disorder, attention deficit hyperactivity disorder, cancer, diabetes, AIDS, and HIV.

Briefly described according to one embodiment of the present invention, a natural composition is disclosed, wherein natural composition is adaptively formulated to modulate multiple pathophysiological processes in order to facilitate homeostatic glucose metabolism, and more particularly to increase insulin sensitivity. The natural composition is a safe and effective formulation for the etiological prevention and treatment of diseases, complications, conditions, and/or disorders associated with chronic or long-term destabilization of glucose metabolism.

The natural composition comprises vitamins, fatty acids, amino acids, phytochemicals, trace minerals, antioxidants, ADP-to-ATP conversion inducer, and metals. The natural composition is adapted so as to be administered in solid or liquid form, thus allowing for various means or systems to be utilized for delivering the natural composition. The delivery systems include but are not limited to oral administration, injection, topical, and nasal spray.

The natural composition is envisioned to further comprise cell messenger agents and growth factors.

The use of the present invention provides an etiological treatment and prevention for diseases, complications, conditions, or disorders associated with chronic or long-term destabilization of glucose metabolism, and particularly insulin resistance in a manner which is quick, easy, and efficient.

### BRIEF DESCRIPTION OF THE DRAWINGS

The advantages and features of the present invention will become better understood with reference to the following more detailed description and claims.

### DESCRIPTION OF THE PREFERRED EMBODIMENT

### 1. Detailed Description

A natural composition is disclosed, according to the present invention, being adaptively formulated to modulate multiple pathophysiological processes in order to facilitate homeostatic glucose metabolism, and more particularly to increase insulin sensitivity. The natural composition is a safe and effective formulation for the etiological prevention and treatment of diseases, complications, conditions, or disorders associated with chronic or long-term destabilization of glucose metabolism.

### 2. Composition

The natural composition is comprised of the following ingredients: vitamins; fatty acids; amino acids; phytochemicals; trace minerals; antioxidants; and metals. The natural composition is adapted so as to be administered in solid or liquid form, thus allowing for various means or systems to be utilized for delivering the natural composition. The delivery systems include but are not limited to oral administration, injection, topical, and nasal spray.

A. Vitamins

Vitamins are nutrients required for essential metabolic reactions in the body. Vitamins can act both as catalysts and substrates in chemical reactions. Vitamins help to convert fat and carbohydrates into energy, and assist in forming bone and tissue.

Vitamin B-1 (Thiamin) deficiency causes Beriberi, a disease of the nervous system. Symptoms of this disease include emotional disturbances, weight loss, impaired sensory perception, weakness and pain in the limbs, periods of irregular heartbeat, and edema. Heart failure and death may occur in advanced cases.

Vitamin B-2 (Riboflavin) deficiency causes ariboflavinosis. The signs and symptoms of ariboflavinosis include sore throat with redness and swelling of the mouth and throat, mucosa, cheilosis and angular stomatitis (cracking of the lips and corners of the mouth), magenta tongue with atrophy, seborrheic dermatitis (moist, scaly skin particularly affecting the scrotum or labia majora and the nasolabial folds), and a decreased red blood cell count with normal cell size and hemoglobin content.

Vitamin B-3 (Niacin) deficiency causes pellagra. Symptoms of pellagra include dermatitis, insomnia, weakness, aggression, mental confusion, and diarrhea. In advanced cases, pellagra may lead to dementia and death.

Vitamin B-5 (Pantothenic acid) deficiency can result in acne and paresthesia. Paresthesia is a sensation of tingling, pricking, or numbness of a person's skin.

Vitamin B-6 (Pyridoxine) deficiency may lead to depression, dermatitis, high blood pressure, anemia, and elevated levels of homocysteine.

Vitamin B-9 (Folic Acid) deficiency results in elevated levels of homocysteine. Folic acid deficiency result in the following symptoms: diarrhea, appetite loss, weight loss, sore tongue, headaches, weakness, heart palpitations, irritability, and behavioral disorders. Pregnant women with folate deficiency are more likely to give birth to premature and low birth weight infants and infants with neural tube defects.

Vitamin B-12 (Cyanocobalamin) deficiency results in fatigue, anemia, weakness, constipation, appetite loss, weight loss, neurological changes such as paresthesia, depression, difficulty in maintaining balance, confusion, poor memory, dementia, and soreness of the mouth or tongue. Symptoms or health problems associated with vitamin B-12 deficiency in infancy include mobility disorders, delayed development, failure to thrive, and megaloblastic anemia.

Vitamin D (Calciferol) is a hormone precursor that contributes to the maintenance of normal levels of calcium and phosphorus in the blood. Vitamin D deficiency is know to cause several bone diseases such as rickets, osteoporosis, and osteomalacia.

Vitamin H (Biotin) is utilized in cell growth, production of fatty acids, metabolism of fats, and amino acids. Biotin also plays a role in the Krebs Cycle which is the process in which energy is released from food. Biotin not only assists in various metabolic chemical conversions, but also helps with the transfer of carbon dioxide. Biotin further aids in maintaining a steady blood sugar level.

Initial symptoms or health problems associated with biotin deficiency include dry skin, seborrheic dermatitis, fungal infections, rashes including erythematous periorofacial macular rash, fine and brittle hair, hair loss or total alopecia. If biotin deficiency is left untreated, neurological symptoms can develop which include, mild depression, which may progress to profound lassitude and eventually to somnolence, generalized muscular pains, changes in mental status, hyperesthesias and paresthesias.

The composition comprises one or more vitamins, wherein vitamins include vitamin B-1, vitamin B-2, vitamin B-3, vitamin B-5, vitamin B-6, vitamin B-9, vitamin D, and vitamin H. The concentration of vitamin B-1 is an amount ranging from about 10 mg to 2000 mg. The concentration of vitamin B-2 is an amount ranging from about 10 mg to 2000 mg. The concentration of vitamin B-3 is an amount ranging from about 10 mg to 2000 mg. The concentration of vitamin B-5 is an amount ranging from about 10 mg to 2000 mg. The concentration of vitamin B-6 is an amount ranging from about 10 mg to 2000 mg. The concentration of vitamin B-9 is an amount ranging from about 0.1 mg to 50 mg. The concentration of vitamin B-12 is an amount ranging from about 0.005 mg to 0.1 mg. The concentration of vitamin D is an amount ranging from about 0.001 mg to 0.020 mg. The concentration of vitamin H is an amount ranging from about 0.1 mg to 1 mg.

B. Fatty acids

Alpha-Lipoic Acid

Alpha-lipoic acid is a redox-active molecule capable of thiol-disulfide exchange, giving it antioxidant activity. Alpha-lipoic acid also scavenges reactive oxygen species and reduces other metabolites, thereby maintaining a healthy cellular redox state. The main function of alpha-lipoic acid is to increase the body's production of glutathione which helps dissolve toxic substances in the liver. Alpha-lipoic acid has been shown in cell culture experiments to increase cellular uptake of glucose by recruiting the glucose transporter to the cell membrane. Studies of rodent aging have suggested that the use of alpha-lipoic acid results in improved memory performance and delayed structural mitochondrial decay.

Alpha-lipoic acid has been shown recently in various clinical trials to improve insulin-mediated glucose metabolism in Type II diabetic subjects. Improvements in insulin-stimulated glucose transport in insulin-resistant rat skeletal muscle and cardiac muscle have been demonstrated following both acute and chronic treatments with alpha-lipoic acid. Alpha-lipoic acid has also been shown to have a beneficial role in the prevention and treatment of neurodegenerative disorders, including the improvement of symptoms of diabetic polyneuropathy, a condition associated with oxidative damage. An essential fatty acid and a prostaglandin precursor, linolenic acid is also known to improve various indexes of diabetic polyneuropathy. Recently, combined treatment with alpha-lipoic acid and linolenic acid, either as individual components or as a conjugate, has been demonstrated to have a synergistic effect in improving neurovascular function in diabetic rats.

The composition comprises alpha-lipoic acid, wherein the concentration of alpha-lipoic acid is an amount ranging from about 5 mg to 50 mg.

Conjugated Linoleic Acid

Linoleic acid (LA) is a naturally occurring fatty acid found predominantly in beef and dairy products. LA is one of the two essential fatty acids (the other being linolenic acid). Linoleic acid is an omega-6 fatty acid, meaning that it is unsaturated, with a double bond occurring at the sixth carbon atom from the omega end of the molecule. Conjugated linoleic acid (CLA) is an isomer of LA, which refers to a slight rearrangement of the molecular structure or a conjugation, resulting in a fatty acid with altered chemical functions. The rearrangement in this case is a conjugated double bond occurring at carbons 10 and 12 or at carbons 9 and 11. CLA is found primarily in meat and dairy products.

CLA is theorized to exhibit anti-tumor/anti-cancer properties. CLA is also theorized to modulate the production of prostaglandins, which are derived from fatty acid molecules and have been linked to an elevated synthesis of growth hormone. Some prostaglandins may also increase blood circulation to the muscles and adipose tissue, the effect of which is believed to improve muscle function and fat mobilization.

The majority of research on dietary intake of CLA has been conducted in animals, wherein the studies derived therefrom have indicated an anti-tumor effect of CLA in doses ranging from 1-4 grams. The potential anti-cancer effects of CLA (most notable the cis-9/trans-11 isomer) have been attributed to several possible mechanisms including CLA's actions as an antioxidant.

Positive effects of CLA on body composition have been shown in numerous animal studies performed on mice, pigs, rats, chicks, and livestock. The studies have revealed that the animal subjects supplemented with CLA consistently lead to animals gaining less body fat, but more lean body tissue, or muscle. In addition, in livestock studies, supplemental CLA has shown to promote growth and prevent muscle atrophy. Still further, CLA supplementation has revealed a reduction in low-density lipoprotein (LDL) cholesterol and triglyceride levels in rabbits with elevated cholesterol.

The composition comprises CLA, wherein the concentration of CLA is an amount ranging from about 50 mg to 800 mg.

C. Amino Acids

L-Arginine

L-arginine is the precursor of endogenous nitric oxide (NO) and a potent stimulator of pituitary growth hormone and pancreatic insulin secretion which directly supports insulin binding to L-arginine's receptor. Both growth hormone and insulin have vasodilatory effects which are mediated via NO. L-arginine also increases insulin receptor sensitivity via constitutive Nitric Oxide (NO). NO is a key substance in the endogenous defense against vascular occlusion and thrombosis, as well as in improving the dynamic and theological vascular responses in patients with Type II diabetes mellitus and/or insulin resistance syndrome. L-arginine further aids in decreasing blood viscosity and platelet aggregation.

The composition comprises L-arginine in an amount ranging from about 65 mg to 3250 mg.

L-Carnitine

L-carnitine transports long-chain fatty acids across the inner mitochondrial membranes in the mitochondria, where they are processed by beta-oxidation to produce biological energy in the form of ATP. L-carnitine deficiency can lead to reduced fatty acid oxidation and limited ATP production. Because the heart depends upon fatty acids as its primary fuel, it is extremely important that the body maintains normal levels of L-carnitine. Where the heart lacks sufficient fatty acids for transport into the mitochondria via carnitine to provide its energy requirement, glucose is utilized as a substitute energy source. However, glucose may not supply sufficient energy for normal cardiac function, particularly in progressive Type II diabetes mellitus and insulin resistance, thus potentially leading to cardiac arrest and death. L-carnitine deficiency can also lead to excessive exposure by tissues to fatty acids which is one of the causes of progressive insulin resistance.

The composition comprises L-carnitine in an amount ranging from about 75 mg to 2700 mg.

L-Taurine

L-taurine modulates cell membrane stabilization and levels of cellular Ca²+. L-taurine drives cytosolic Ca²+ into the mitochondria where Ca²+ is involved in the mitochondrial production of ATP. Thereafter, ATP exits the mitochondria to supply energy in order to pump cytosolic Ca²+ out of the cell.

L-taurine is also an osmoregulator, functioning to ameliorate diabetic neuropathy and nephropathy. L-taurine further stabilizes platelet membranes, reduces platelet aggregation, and has been shown to reduce cellular insulin resistance.

The composition comprises L-taurine in an amount ranging from about 50 mg to 3125 mg.

L-Alanine

Research has demonstrated that L-alanine is a strong stimulus to insulin secretion in the presence of glucose in β-cell lines. It has been further demonstrated that L-alanine dramatically enhances nuclear magnetic resonance measurable aspects of glucose metabolism (both oxidative and nonoxidative). The presence of L-alanine has also been shown to enhance the rate of entry of glucose-derived pyruvate into the tricarboxylic acid cycle which in turn stimulated rates of generation of key metabolites, such as ATP, thereby affecting the insulin secretory process.

The composition comprises L-alanine in an amount ranging from about 60 mg to 3000 mg. The composition further comprises L-leucine, L-valine, and L-isoleucine, wherein the concentration of L-leucine is in an amount ranging from about 25 mg to 1000 mg, the concentration of L-valine is in an amount ranging from about 20 mg to 1100 mg, and the concentration of L-isoleucine is an amount ranging from about 30 mg to 1500 mg.

D. Phytochemicals

Phytochemicals are substances that are naturally occurring only in plants. It has been demonstrated that phytochemicals may provide health benefits beyond those provided by essential nutrients, such as vitamins and minerals. Eating a variety of colorful phytochemical-rich fruits and vegetables has been associated with a lower risk of chronic diseases including cancer and heart disease. Phytochemicals also act as antioxidants. Phytochemicals further protect and regenerate essential nutrients and/or work to deactivate cancer-causing substances. It is theorized that phytochemicals, vitamins, minerals, and fiber operate synergistically to promote health and lower disease risk.

Siberian Eleuthero Root

Siberian eleuthero root contains active chemical ingredients called eleutherosides which have been shown to produce moderate reductions in blood sugar and blood cholesterol levels and modest improvements in memory and concentration. Siberian eleuthero root may also have estrogenic effects, and appears to boost immune system function. In laboratory studies, eleutherosides have been shown to provide antiviral and anti-cancer properties. Additional research has shown that siberian eleuthero root also stimulates resistance to stress.

The composition comprises siberian eleuthero root in an amount ranging from about 50 mg to 850 mg.

Astragalus Membranaceus

Astragalus membranaceus is a tonic herb theorized to increase lactation. Recent studies have shown that astragalus membranaceus strengthens the human immune system and increases the body's resistance to common viruses. Astragalus membranaceus is further theorized to aid in the healing of wounds and injuries.

The composition comprises astragalus membranaceus in an amount ranging from about 40 mg to 750 mg.

Ginseng

For purposes of this disclosure, ginseng is intended to include panax ginseng (Chinese/Korean ginseng root) and panax quinquefolius (American ginseng root).

Ginseng is a powerful adaptogen. An adaptogen is a nontoxic substance that helps to increase resistance against stressful influences of a physical, chemical, or biological nature, and in general, has a normalizing effect. These attributes are believed to be achieved from ginseng's ability to stimulate functions that regulate the central nervous system, the cardiovascular system, and the endocrine glands. Studies have shown that ginseng counteracts effects of physical and emotional stress, stimulates the immune system, spurs production of the body's own virus-fighting chemicals, enhances memory, helps reduce cholesterol levels in the blood, helps control diabetes by reducing sugar levels, has anti-clotting effects, thereby reducing the risk of arterial blood clots, serves as an antioxidant, thereby preventing cumulative damage believed to culminate into cancer, minimizes cell damage from radiation, counteracts fatigue without caffeine and improves stamina, protects the liver from the effects of drugs, alcohol and toxins, and increases intestinal absorption of nutrients.

In addition, clinical tests with elderly patients having high blood pressure showed that ginseng supplementation produced a reduction in blood pressure.

Further, ginseng is theorized to have positive effects on the sexual glands as one portion of its overall effect on the hormones of the body. Ginseng is also theorized to reduce and prevent side effects associated with menopause due to ginseng's ability to regulate hormones.

The composition comprises ginseng in an amount ranging from about 10 mg to 1000 mg.

Acerola Berry Powder

Acerola berry contains the most potent source of natural vitamin C and bioflavanoids. Acerola berry has been demonstrated to be very useful in the colon where it helps to balance pH levels to allow for proper colon function. Acerola berry has further been shown to protect and preserve the structure of capillary blood vessels, to help promote circulation, and to stimulate bile production in the liver.

The composition comprises acerola berry powder in an amount ranging from about 50 mg to 900 mg.

Cinnamon

Research data has shown that cinnamon contains an active compound, methylhydroxy chalcone polymer (MHCP) that increased glucose metabolism roughly 20-fold in a test tube assay of fat cells. Research has further demonstrated that MHCP prevented the formation of damaging oxygen radicals in a blood platelet assay.

A recent study (2003) performed on rats demonstrated cinnamon's beneficial effects on insulin activity. In this study, rats were given a daily dose of cinnamon (300 mg per kilogram of body weight) for a three week period which resulted in the rats' skeletal muscle being able to absorb 17% more blood sugar per minute compared to that of control rats, which had not received cinnamon. Researchers attributed this increase to cinnamon's enhancement of the muscle cells' insulin-signaling pathway. In humans with Type II diabetes mellitus, consuming as little as 1 gram of cinnamon per day was found to reduce blood sugar, triglycerides, LDL cholesterol, and total cholesterol, in a study published in 2003.

A placebo-controlled study (2004) evaluated sixty people with Type II diabetes mellitus (30 men and 30 women ranging in age from 44 to 58 years) who were divided into six groups. Groups 1, 2, and 3 were given 1, 3, or 6 grams of cinnamon daily, while groups 4, 5, and 6 received 1, 3, or 6 grams of placebo. After 40 days, all three levels of cinnamon reduced blood sugar levels by 18-29%, triglycerides 23-30%, LDL cholesterol 7-27%, and total cholesterol 12-26%, while no significant changes were seen in those groups receiving placebo. The researchers concluded that cinnamon in the diet of people with Type II diabetes mellitus will reduce risk factors associated with diabetes and cardiovascular diseases.

Cinnamon also provides other unique healing abilities which are derived from three basic types of components in the essential oils found in its bark. These oils contain active components called cinnamaldehyde, cinnamyl acetate, and cinnamyl alcohol. Cinnamaldehyde helps prevent unwanted clumping of blood platelets by inhibiting the release of an inflammatory fatty acid called arachidonic acid from platelet membranes and reducing the formation of an inflammatory messaging molecule called thromboxane A2. Cinnamon's ability to lower the release of arachidonic acid from cell membranes also characterizes it as an anti-inflammatory.

In addition, cinnamon's essential oils also allow it to be characterized as an anti-microbial. Cinnamon has been studied for its ability to help stop the growth of bacteria as well as fungi, including the commonly problematic yeast Candida. In laboratory tests, growth of yeasts that were resistant to the commonly used anti-fungal medication fluconazole was often stopped by cinnamon extracts.

The composition comprises cinnamon in an amount ranging from about 100 mg to 1000 mg.

Flax Powder or Lignans

Flax powder is made from pure ground flax seed. Flax powder is rich in lignans. Lignans are a phytonutrient found in both seeds and grains and are a type of carbohydrate. Lignans are a great source of healthy omega-3 fatty acids.

Flaxseed yields over 800 mcg/g of lignans whereas flaxseed oil has fewer than 2% of lignans. The lignans of flaxseed oil is a phenolic compound or polyphenol called secoisolariciresinal glucoside. The National Cancer Institute recognizes secoisolariciresinal glucoside's ability to prevent cancer. In addition to having anti-cancer properties, secoisolariciresinal glucosides also have anti-viral, anti-bacterial and anti-fungus properties. Lignans have also been shown to increase immune system function, thereby being effective against a number of diseases.

Lignans contain phytoestrogens that mimic estrogen in the body and can be used as an alternative to hormone replacement therapy. Increased phytoestrogens have the potential to lower breast and colon cancer. Recent research indicates that lignans may be capable of inhibiting the formation, of tumors. Consistent use of flaxseed lignans has further shown the ability to increase bowel function up to 30% among other benefits.

The composition comprises flaxseed powder in an amount ranging from about 5 mg to 1000 mg, or lignans in an amount ranging from about 0.004 mg to 0.8 mg or from about 4 mcg to 800 mcg.

Spirulina

Spirulina contains unusually high amounts of protein, specifically between 55% and 77% dry weight. Spirulina is a complete protein, comprising all essential amino acids. However, spirulina contains reduced amounts of cysteine, methionine, and lysine as compared to standard proteins such as from milk, eggs, or meat.

Spirulina is a rich source of gamma-linolenic acid and also provides alpha-linolenic acid, linoleic acid, stearidonic acid, eicosapentaenoic acid, docosahexaenoic acid, and arachidonic acid.

Spirulina also contains vitamins, namely vitamins B-1, B-2, B-3, B-6, B-9, B-12, C, D, and E. Spirulina is a rich source of potassium and additionally contains calcium, chromium, copper, iron, magnesium, manganese, phosphorus, selenium, sodium, and zinc.

Spirulina contains many pigments including myxoxanthophyll, zeaxanthin, chlorophyll-a, beta-carotene, canthaxanthin, xanthophyll, echinenone, diatoxanthin, beta-cryptoxanthin, 3′-hydroxyechinenone, in addition to the phycobilins allophycocyanin and c-phycocyanin.

In vitro research has shown that spirulina extract inhibits HIV replication in human T-cells, peripheral blood mononuclear cells and Langerhans cells (1998).

Animal research has shown the following: spirulina helps prevent heart damage caused by chemotherapy using Doxorubicin, without interfering with its anti-tumor activity (2005); spirulina reduces the severity of strokes and improves recovery of movement after stroke (2005); spirulina reverses age-related declines in memory and learning (2002); and spirulina prevents and treats hay fever (2005).

Clinical trials have demonstrated the following: spirulina is effective for the clinical improvement of melanosis and keratosis due to chronic arsenic poisoning (2006); spirulina improves wight-gain and corrects anemia in both HIV-infected and

HIV-negative undernourished children (2005); and spirulina protects against hay fever (2005).

The composition comprises spirulina in an amount ranging from about 20 mg to 1200 mg.

E. Trace Minerals

Trace minerals are dietary minerals needed by the human body in very small quantities.

Chromium

Chromium is an essential nutrient involved in the metabolism of glucose, insulin and blood lipids. Suboptimal dietary intake of chromium is associated with increased risk factors associated with diabetes and cardiovascular diseases. Chromium is a cofactor for insulin—it increases insulin binding to cells, insulin receptor number and activates insulin receptor kinase leading to increased insulin sensitivity. In addition, double-blind, placebo-controlled studies have demonstrated that, as part of a healthy diet and exercise program, chromium supplementation can help reduce body fat, increase or maintain lean body tissue and can lead to weight loss.

The composition comprises chromium, wherein chromium is in the form of a member or combination thereof selected from the group consisting of chromium picolinate, chromium nicotinate, chromium dinicotinate, chromium tripicolinate, and chromium polynicotinate. The concentration of chromium picolinate is an amount ranging from about 100 mcg to 1200 mcg. The concentration of chromium nicotinate is an amount ranging from about 100 mcg to 1200 mcg. The concentration of chromium dinicotinate is an amount ranging from about 100 mcg to 1200 mcg. The concentration of chromium tripicolinate is an amount ranging from about 100 mcg to 1200 mcg. The concentration of chromium polynicotinate is an amount ranging from about 25 mcg to 1200 mcg.

Vanadium

Research over the past century, which included pre-clinical laboratory trials and clinical studies, validated that vanadium may activate insulin receptors, and through this effect exert insulin-like action. Both pre-clinical and clinical studies have demonstrated that vanadium has a potential role in the management of both Type I and Type II diabetes. Vanadium's insulin-like behavior appears to improve glucose management in insulin-dependent diabetics. In Type II diabetics, the mineral improves glucose tolerance and lowers blood glucose levels. Vanadium has also been shown to inhibit the production of cholesterol. Thus, vanadium supplementation has a potential role in maintaining blood sugar levels in diabetics.

The composition comprises vanadium in an amount ranging from about 1 mg to 400 mg.

Zinc

Zinc plays a significant role in the synthesis, storage, and secretion of insulin as well as conformational integrity of insulin in the hexameric form. The relationship between diabetes, insulin and zinc is complex. Diabetes effects zinc homeostasis in many ways, particularly, hyperglycemia, which is responsible for the increased urinary loss and decreases in total body zinc. Zinc deficiency exacerbates the cytokine-induced damage in the autoimmune attack which destroys the islet cell in Type I diabetes, thereby preventing the islet cell from producing and secreting insulin. Insulin is secreted by the β-cell both tonically and as a high level spike in response to an immediate glucose load such as a meal. It has been known for decades that a physical chemical relationship exists between insulin and zinc. The addition of zinc to insulin changes the time course of the effect of a given dose of insulin. In the presence of zinc within the islet cell, insulin monomers assemble to a dimeric form for storage and secretion as a zinc crystal. Research has shown that high concentrations of glucose and other secretagogues decrease the islet cell labile zinc and video fluorescence analysis has shown that zinc concentrated in the islet cells was related to the synthesis, storage, and secretion of insulin.

It is also clear that there is loss of a large amount of zinc from the body via the urine. It has been postulated that hyperglycemia interferes with the active transport of zinc back into the renal tubular cells.

Concerning the effects of zinc on diabetes mellitus, zinc has been found to have multiple roles which include an inhibition of the post insulin receptor intracellular events which results in a decreased glucose tolerance, and a relative decrease in insulin secretion, since the elevated glucose should produce a more exuberant insulin response. This suggests that zinc deficiency also reduces the ability of the pancreas to respond appropriately.

The composition comprises zinc in an amount ranging from about 0.5 mg to 100 mg.

The composition further comprises additional trace minerals which include iodine, copper, manganese, and molybdenum. The concentration of iodine is an amount ranging from about 25 mcg to 200 mcg. The concentration of copper is an amount ranging from about 0.5 mg to about 4 mg. The concentration of manganese is an amount ranging from about 0.5 mg to about 4 mg. The concentration of molybdenum is an amount ranging from about 25 mcg to 125 mcg.

F. Antioxidants

Antioxidants are instrumental in neutralizing free radicals in the body that cause chronic disease.

Tocopherol

Tocopherol (Vitamin E) is a fat-soluble vitamin existing in eight forms or isomers. The isomers include four tocopherols and four tocotrienols. All isomers include a chromanol ring with a hydroxyl group which donates a hydrogen atom to reduce free radicals and a hydrophobic side chain which allows for penetration into biological membranes.

α-tocopherol is traditionally recognized as the most active form of vitamin E in humans. Because diabetes has been shown to increase oxidative stress and because cardiovascular complication is among the leading causes of death in diabetics, α-tocopherol supplementation of individuals afflicted with diabetes is envisioned.

A recent study found a biochemical marker of oxidative stress to be elevated in diabetic individuals. Supplementation with 600 mg of synthetic α-tocopherol daily for 14 days resulted in a reduction in the oxidative stress marker. (See Davi G, Ciabattoni G, Consoli A, et al. In vivo formation of 8-iso-prostaglandin f2alpha and platelet activation in diabetes mellitus: effects of improved metabolic control and vitamin E supplementation. Circulation. 1999; 99(2):224-229).

The composition comprises tocopherol, wherein tocopherol includes α-tocopherol in an amount ranging from about 5 mg to 500 mg, and preferably RRR-α-tocopherol or all-racemic a-tocopherol in an amount ranging from about 2.5 mg to 400 mg.

Beta-carotene

Beta-carotene is derived from the Latin name for carrot and belongs to a family of natural chemicals known as carotenoids or carotenes. Carotenoids are widely found in plants and are what give yellow and orange fruits and vegetables their vibrant colors. Beta-carotene is converted to vitamin A or retinol by the body. Some studies suggest that dietary intake of beta-carotene may reduce the risk of heart disease and cancer.

The composition comprises beta-carotene in an amount ranging from about 10 mg to 50 mg, wherein the envisioned daily dosage for pediatric use is in an amount ranging from about 10 mg to 20 mg, and the envisioned daily dosage for adult use is in an amount ranging from about 15 mg to 50 mg.

Ubiguinone

Ubiquinone or co-enzyme Q10 is found in the membranes of endoplasmic reticulum, peroxisomes, lysosomes, vesicles, and the inner membrane of the mitochondrion where it is an important part of the electron transport chain that leads to ATP generation. Ubiquinone is involved in direct electron transfer towards oxygen and is also essential in the formation of the apoptosome along with other adapter proteins. A loss of trophic factors activates pro-apoptotic enzymes, causing breakdown of mitochondria. In addition to the production of ATP and maintenance of cellular and mitochondrial membrane fluidity, ubiquinone is an effective remover of free radicals.

Diabetics have a deficiency of ubiquinone. Ubiquinone deficiency in the pancreas can impair the generation of ATP and the biosynthesis of insulin. Research trials have suggested that oral treatment with ubiquinone or co-enzyme Q10 is effective in decreasing blood pressure and improving insulin response.

The composition comprises ubiquinone or co-enzyme Q-10 in an amount ranging from about 3 mg to 300 mg.

Lycopene

Lycopene is a carotenoid found in red fruits such as tomatoes and watermelons. Carotenoids are natural pigments that act as powerful antioxidants in the body. Lycopene has been shown to neutralize free radicals, particularly those derived from oxygen, thereby conferring protection against diabetes, osteoporosis, male infertility, prostate cancer, breast cancer, atherosclerosis, and associated coronary artery disease. Lycopene also reduces LDL oxidation and helps reduce cholesterol levels in the blood. Current research is examining the affect of lycopene on such conditions as macular degnerative disease and serum lipid oxidation.

The composition comprises lycopene in an amount ranging from about 0.25 mg to 1.25 mg.

Ascorbic Acid

Diabetics have less concentrations of ascorbic acid (vitamin C). Insulin promotes the cellular uptake and level of ascorbic acid, while insulin resistance and hyperglycemia reduces the same. Ascorbic acid is a cofactor of hydroxylation in carnitine biosynthesis. Persons having below normal ascorbic acid concentrations have reduced levels of carnitine. Fatigue and weakness typically prevalent in progressive insulin resistance and Type II diabetes mellitus is believed to be directly related to carnitine deficient mitochondrial dysfunction, wherein such mitochondrial dysfunction being indirectly due to insufficient cytosolic ascorbic acid.

Ascorbic acid has been shown to significantly decrease cellular insulin glycation. In addition, ascorbic acid is a powerful antioxidant in the cytosol, a cofactor in collagen biosynthesis, and a platelet activation inhibitor, polyol pathway inhibitor, and prostaglandin synthesis inhibitor, wherein the inhibition of prostaglandin synthesis facilitates a reduction in microvascular permeability and nonenzymatic protein glycation.

Hyperglycemia actuates oxidative free radical stress when free radicals are release from the auto-oxidation of glucose. Absorbic acid is highly consumed in diabetics, thereby necessitating ascorbic acid supplementation.

The composition comprises ascorbic acid (vitamin C) in an amount ranging from about 60 mg to 3200 mg.

Tetrahydrobiopterin

Tetrahydrobiopterin (BH4) is an essential cofactor required for activity of nitric oxide synthases. Existing evidence suggests that, during activation of constitutive and inducible isoforms of nitric oxide synthase, tetrahydrobiopterin is needed for allosteric and redox activation of enzymatic activity. However, precise mechanisms underlying the role of tetrahydrobiopterin in regulation of nitric oxide formation is not fully understood. In cerebral and peripheral arteries, increased availability of tetrahydrobiopterin can augment production of nitric oxide. In contrast, in arteries depleted of tetrahydrobiopterin, production of nitric oxide is impaired. The ability of vascular tissues to synthesize tetrahydrobiopterin plays an important role in regulation of nitric oxide synthase under physiological conditions as well as during inflammation and sepsis. Recent studies concerning expression and function of recombinant nitric oxide synthases suggest that availability of tetrahydrobiopterin is important for production of nitric oxide.

The composition comprises tetrahydrobiopterin in an amount ranging from about 25 mg to 3250 mg.

N-acetylcysteine

Research has shown and it has been observed that Glutathione (GSH) administration increases insulin sensitivity in diabetic patients due to its antioxidant properties. GSH is known chemically as N-(N-L-gamma-glutamyl-L-cysteinyl) glycine. GSH is the paramount intracellular defense against free radicals produced via mitochondrial metabolism and excess free radicals via hyperglycemia. L-cysteine and N-acetylcysteine are precursors of GSH.

GSH is a tripeptide that cannot be adequately absorbed from the gastrointestinal tract and requires a substrate, such as N-acetylcysteine. In addition, selenium is an essential cofactor for glutathione peroxidase, which is required in order to facilitate optimal GSH activity.

GSH is synthesized in two adenosine triphosphate (ATP)-dependent steps. In the first step, gamma-glutamylcysteine is synthesized from L-glutamate and cysteine via the enzyme gamma-glutamylcysteine synthetase. The liver is the principal site of glutathione synthesis. In healthy tissue, more than 90% of the total glutathione pool is in the reduced form and less than 10% exists in the disulfide form. The enzyme glutathione disulfide reductase is the principal enzyme that maintains glutathione in its reduced form. Glutathione disulfide reductase utilizes nicotinamide adenine dinucleotide phosphate (NADPH) as its cofactor. NADPH is generated by the oxidative reaction in the pentose phosphate pathway.

GSH is diminished in diabetics. The consequences of a functional glutathione deficiency, which results in tissue oxidative stress, can be seen in particular pathological conditions. For example, persons with glucose 6-phosphate dehydrogenase deficiency produce lower amounts of NADPH, hence the lower amounts of reduced glutathione. This condition is characterized by a hemolytic anemia. Conditions causing chronic glutathione deficiency all result in hemolytic anemia, among other pathological consequences. Oxidative stress caused by glutathione deficiency results in fragile erythrocyte membranes. Chronic functional glutathione deficiency is also associated with immune disorders, an increased incidence of malignancies, and in the case of HIV disease, probably accelerated pathogenesis of the disease. Glutathione has also been shown to enhance insulin secretion in elderly subjects with impaired glucose tolerance.

Oral administration of N-acetylcysteine stimulates GSH synthesis, thereby enhancing levels of GSH in the liver, plasma, and in the bronchoalveolar lavage fluid. Oral administration of N-acetylcysteine further enhances glutathione-S-transferase activity, promotes detoxification, and acts directly on reactive oxidant radicals. Thus, oral administration of an effective dose of N-acetylcysteine increases insulin sensitivity or reduces insulin resistance.

The composition comprises N-acetylcysteine in an amount ranging from about 90 mg to 50 g. In addition, the composition further comprises selenium in an amount ranging from about 0.015 mg to 1 mg.

It is contemplated the composition comprises a high oxygen radical absorbance capacity blend (ORAC). ORAC is comprised of berry extracts which include strawberry extract, blueberry extract, blackberry extract, cranberry extract, and raspberry extract.

The composition comprises ORAC in an amount ranging from about 5 mg to 100 mg.

G. Metals

Magnesium

Magnesium (Mg2+) levels are significantly lowered in diabetics, and lowest in those with severe retinopathy. Studies suggest that a deficiency in magnesium may worsen the blood sugar control in Type II diabetes mellitus. Scientists believe that a deficiency of magnesium interrupts insulin secretion in the pancreas and increases insulin resistance in the body's tissues.

The American Diabetes Association recommends that all patients with normal renal function who have diabetes mellitus to receive magnesium supplementation. Importantly, patients with insulin resistance and Type II diabetes mellitus, circulating insulin induces an increase in the renal excretion of magnesium.

Research scientists believe that a magnesium deficient state causes insulin resistance, with the plasma magnesium level inversely related to insulin sensitivity. Thus, research indicates that magnesium supplementation should improve both insulin sensitivity and insulin secretion in patients with Type II diabetes mellitus.

Supplementation with magnesium has demonstrated improved insulin production in elderly people with Type II diabetes mellitus. Elders without diabetes may also produce more insulin as a result of magnesium supplements. Insulin requirements are lower in people with Type I diabetes who supplement with magnesium.

Diabetes-induced damage to the eyes is more likely to occur to magnesium-dificient people with Type I diabetes. In pregnant women with Type I diabetes who were magnesium deficient, the lack of magnesium may even account for the high rate of spontaneous abortion and birth defects associated with Type I diabetes. Low magnesium levels appear to be a significant risk factor in the development of cardiovascular disease, particularly coronary artery spasm.

The composition comprises magnesium in an amount ranging from about 15 mg to 1100 mg.

Calcium

Changes in cytosolic free calcium concentration (Ca2+) constitute an important element of signal transduction in various cells. These changes reflect either alterations in calcium fluxes or result from mobilization of intracellular stores. In pancreatic islet cells, an increase in calcium is critical for secretagogue-induced insulin release. Thus, glucose evokes a rapid increase in calcium, primarily by stimulating calcium influx. Under physiologic conditions, glucose may also promote mobilization of intracellular calcium stores via stimulating membrane phospholipid hydrolysis and formation of inositol triphosphate. Inositol triphosphate is a potent stimulus for calcium mobilization. In order for glucose to engage in this activity, it

requires the presence of extracellular calcium. The magnitude of change in calcium may not coincide with the level of insulin release, thereby conveying that the role of calcium in the process of insulin release must be considered together with other cellular mechanisms.

Recent research has indicated that when calcium concentrations are too low or too high, the ability of pancreatic islets and insulin target cells to respond appropriately to physiologic stimuli is substantially diminished. Impaired cellular calcium homeostasis may represent an essential link in the pathogenesis of impaired insulin secretion and in the pathogenesis of impaired insulin action.

The composition comprises calcium in an amount ranging from about 5 mg to 750 mg.

It is contemplated that the natural composition further comprises cell messenger agents which include inositol and choline.

Inositol

Inositol is a fundamental ingredient of cell membranes and is necessary for proper nerve, brain, and muscle function. Inositol is lipotropic and functions conjunctively with vitamins B-6, B-12, choline, folacin, betaine, and methionine to prevent the accumulation of fats in the liver.

Diabetic neuropathy is a nerve disease caused by diabetes. The loss of inositol from the nerve cell is a major cause of the decrease nerve function. Research performed in 1983 revealed that inositol supplements may improve nerve conduction velocities in diabetics.

Inositol compounds have also demonstrated remarkable qualities in the prevention and treatment of cancer. According to recent research, inositol increases the differentiation and normalization of cancer cells. Fiber is amply supplied with inositol hexaphosphate which may partially explain why high-fiber diets are associated with a lower incidence of certain cancers.

Furthermore, neurotransmitters such as serotonin and acetylcholine in the brain depend on inositol to function properly. Low levels of this nutrient may result in depression. Increasing inositol levels should provide promising treatment for depressive conditions. Inositol's effect on depression led to a 1995 study designed to test inositol's effectiveness against panic disorder. The study reported that inositol facilitated a reduction in the frequency and severity of panic attacks in patients with panic disorders.

The composition comprises inositol in an amount ranging from about 1 mg to 600 mg.

Choline

Choline is a member of the B-complex vitamins and a component of lecithin. Choline is required for the production of acetylcholine which is an important neurotransmitter. Low levels of acetylcholine are associated with the memory loss of Alzheimer's disease and Huntington's disease. Acetylcholine is essential for the metabolism of fats and the removal of fat from the liver. Acetylcholine must be present in the body for proper function of the nervous system, including mood, behavior, orientation, personality traits, and judgment. Choline deficiency in humans can lead to liver impairment.

In 1998 the government declared choline an essential nutrient and the Food and Drug Administration recently authorized products containing qualifying amounts of choline to carry a nutrient content claim, specifically bearing either good source or excellent source of choline.

Choline is also essential for liver function and research has shown that it has benefits concerning brain development, reproductive development, and cardiovascular health.

Choline further aids in hormone production as well as minimized excess fat storage in the liver. Minimized excess fat storage in the liver stems from choline's ability to aid in fat and cholesterol metabolism. Choline is still further needed for the proper transmission of nerve impulses from the brain through the central nervous system as well as for gallbladder regulation and liver function.

Choline is instrumental in the formation of lecithin. Lecithin is a naturally occurring substance found in every living cell of the body, especially the brain and the liver. Lecithin is a natural source of choline in the form of phosphatidylcholine. Phosphatidylcholine has numerous functions within the body including acting as a powerful fat emulsifier that protects the entire body from excess accumulation of fats.

The composition comprises choline in an amount ranging from about 1 mg to 600 mg.

It is further contemplated that the natural composition also comprises boron, silica, and lutein. The concentration of boron is in an amount ranging from about 0.025 mg to about 50 mg. The concentration of silica is an amount ranging from about 0.025 mg to about 100 mg. The concentration of lutein is an amount ranging from about 100 mcg to about 5 mg.

It is envisioned that the natural composition further comprises growth factors adapted and formulated as an adjunct (homeopathic formulation) to the ingredients described hereinabove to facilitate effective treatment of diseases, complications, conditions, or disorders including diabetes, inflammation, joint and muscle pain, muscle weakness, fatigue, chronic viral infections, cancer, nasal and sinus congestion, respiratory complications, digestive problems, memory loss, neuropathy, and psychological conditions such as depression, mood swings, anger, anxiety, and confusion. The growth factors are further adapted and formulated to aid in reducing body fat and increasing or maintaining lean body tissue, thereby potentially leading to weight loss.

The homeopathic formulation is comprised of one or more growth factors, wherein said homeopathic formulation comprises a molar concentration of between 1×10⁻2 and 1'10⁻1,250,000 of the one or more growth factors. The growth factors include insulin-like growth factor I (IGF-I), insulin-like growth factor II (IGF-II), nerve growth factor (NGF), stem cell growth factor (SCF), fibroblast growth factor (FGF), granulocyte macrophage-colony stimulating growth factor (GM-CSF), granulocyte-colony stimulating growth factor (G-CSF), macrophage-colony stimulating growth factor (M-CSF), transforming growth factor α (TGFα), transforming growth factor β (TGFβ), platelet-derived growth factor (PDGF), tumor necrosis factor a (TNFα), and tumor necrosis factor β (TNFβ).

### 3. Method of Use

The method for the etiological treatment and etiological prevention of diseases, complications, conditions, or disorders associated with chronic or long-term destabilization of glucose metabolism comprises administering a patient an effective amount or dosage of a natural composition. The natural composition comprises vitamins, fatty acids, amino acids, phytochemicals, trace minerals, antioxidants, and metals. The natural composition further comprises cell messenger agents and growth factors.

### 4. Operation of the Preferred Embodiment

To use the present invention, user ingests an effective dosage of the natural composition, wherein the dosage of the natural composition is an amount ranging from about 900 mg to 94.7985 g ingested daily.

The use of the present invention provides an etiological treatment and prevention for diseases, complications, conditions, or disorders associated with chronic or long-term destabilization of glucose metabolism, and particularly insulin resistance in a manner which is quick, easy, and efficient.

Therefore, the foregoing description is included to illustrate the operation of the preferred embodiment and is not meant to limit the scope of the invention. As one can envision, an individual skilled in the relevant art, in conjunction with the present teachings, would be capable of incorporating many minor modifications that are anticipated within this disclosure. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be broadly limited only by the following Claims.

What is claimed is:

1. A natural composition adapted to modulate multiple pathophysiological processes in order to facilitate homeostatic glucose metabolism as a method for the etiological prevention and treatment of diseases, complications, conditions, or disorders associated with chronic or long-term destabilization of glucose metabolism, said natural composition comprising:
(a) vitamins;
(b) fatty acids;
(c) amino acids;
(d) phytochemicals;
(e) trace minerals;
(f) antioxidants; and
(g) metals.

2. The natural composition in accordance with claim 1, wherein said natural composition is adapted so as to be administered in solid or liquid form, thereby allowing for various delivery means or delivery systems to be utilized for delivering said natural composition.

3. The natural composition in accordance with claim 1, wherein said delivery systems include oral administration, injection, topical, and nasal spray.

4. The natural composition in accordance with claim 1, wherein said vitamins comprise:
(a) thiamin;
(b) riboflavin;
(c) niacin;
(d) pantothenic acid;
(e) pyridoxine;
(f) folic acid;
(g) cyanocobalamin;
(h) calciferol; and
(i) biotin.

5. The natural composition in accordance with claim 1, wherein said fatty acids comprise:
(a) alpha-lipoic acid; and
(b) conjugated linoleic acid.

6. The natural composition in accordance with claim 1, wherein said amino acids comprise:
(a) L-arginine;
(b) L-carnitine;
(c) L-taurine;
(d) L-alanine;
(e) L-leucine;
(f) L-valine; and
(g) L-isoleucine.

7. The natural composition in accordance with claim 1, wherein said phytochemicals comprise:
(a) siberian eleuthero root;
(b) astragalus membranaceus;
(c) ginseng;
(d) acerola berry powder;
(e) cinnamon;
(f) flaxseed powder or lignans; and
(g) spirulina.

8. The natural composition in accordance with claim 1, wherein said trace minerals comprise:
(a) chromium;
(b) vanadium; and
(c) zinc.

9. The natural composition in accordance with claim 1, wherein said antioxidants comprise:
(a) tocopherol;
(b) beta-carotene;
(c) ubiquinone;
(d) Iycopene;
(e) ascorbic acid;
(f) tetrahydrobiopterin;
(g) N-acetylcysteine;
(h) selenium; and
(i) a high oxygen radical absorbance capacity blend.

10. The natural composition in accordance with claim 1, wherein said metals comprise:
(a) magnesium; and
(b) calcium.

11. The natural composition in accordance with claim 4, wherein:
(a) said thiamin is in an amount ranging from about 10 mg to 2000 mg;
(b) said riboflavin is in an amount ranging from about 10 mg to 2000 mg;
(c) said niacin is in an amount ranging from about 10 mg to 2000 mg;
(d) said pantothenic acid is in an amount ranging from about 10 mg to 2000 mg;
(e) said pyridoxine is in an amount ranging from about 10 mg to 2000 mg;
(f) said folic acid is in an amount ranging from about 0.1 mg to 50 mg;
(g) said cyanocobalamin is in an amount ranging from about 0.005 mg to 0.1 mg;
(h) said calciferol is in an amount ranging from about 0.001 mg to 0.020 mg; and
(i) said biotin is in an amount ranging from about 0.1 mg to 1 mg.

12. The natural composition in accordance with claim 5, wherein:
(a) said alpha-lipoic acid is in an amount ranging from about 5 mg to 50 mg; and
(b) said conjugated linoleic acid is in an amount ranging from about 50 mg to 800 mg.

13. The natural composition in accordance with claim 6, wherein:
(a) said L-arginine is in an amount ranging from about 65 mg to 3250 mg;
(b) said L-carnitine is in an amount ranging from about 75 mg to 2700 mg;
(c) said L-taurine is in an amount ranging from about 50 mg to 3125 mg;
(d) said L-alanine is in an amount ranging from about 60 mg to 3000 mg;
(e) said L-leucine is in an amount ranging from about 25 mg to 1000 mg;
(f) said L-valine is in an amount ranging from about 20 mg to 1100 mg; and
(g) said L-isoleucine is in an amount ranging from about 30 mg to 1500 mg.

14. The natural composition in accordance with claim 7, wherein:
(a) said siberian eleuthero root is in an amount ranging from about 50 mg to 850 mg;
(b) astragalus membranaceus is in an amount ranging from about 40 mg to 750 mg;
(c) ginseng is in an amount ranging from about 10 mg to 1000 mg;
(d) acerola berry powder is in an amount ranging from about 50 mg to 900 mg;
(e) cinnamon is in an amount ranging from about 100 mg to 1000 mg;
(f) said flaxseed powder is in an amount ranging from about 5 mg to 1000 mg;
(g) said lignans is in an amount ranging from about 4 mcg to 800 mcg; and
(g) said spirulina is in an amount ranging from about 20 mg to 1200 mg.

15. The natural composition in accordance with claim 8, wherein said chromium is in the form of a member or combination thereof selected from the group consisting of chromium picolinate, chromium nicotinate, chromium dinicotinate, chromium tripicolinate, and chromium polynicotinate.

16. The natural composition in accordance with claim 15, wherein:
(a) said chromium picolinate is in an amount ranging from about 100 mcg to 1200 mcg;
(b) chromium nicotinate is in an amount ranging from about 100 mcg to 1200 mcg;
(c) said chromium dinicotinate is in an amount ranging from about 100 mcg to 1200 mcg;
(d) said chromium tripicolinate is in an amount ranging from about 100 mcg to 1200 mcg; and
(e) said chromium polynicotinate is an amount ranging from about 25 mcg to 1200 mcg.

17. The natural composition in accordance with claim 8, wherein:
(a) said vanadium is in an amount ranging from about 1 mg to 400 mg; and
(b) said zinc is in an amount ranging from about 0.5 mg to 100 mg.

18. The natural composition in accordance with claim 9, wherein said tocopherol is comprised of a-tocopherol or all-racemic α-tocopherol.

19. The natural composition in accordance with claim 18, wherein said α-tocopherol is in an amount ranging from about 5 mg to 500 mg.

20. The natural composition in accordance with claim 18, wherein said all-reacemic α-tocopherol is in an amount ranging from about 2.55 mg to 400 mg.

21. The natural composition in accordance with claim 9, wherein:
(a) said beta-carotene is in an amount ranging from about 10 mg to 50 mg;
(b) said ubiquinone is in an amount ranging from about 3 mg to 300 mg;
(c) said lycopene is in an amount ranging from about 0.25 mg to 1.25 mg;
(d) said ascorbic acid is in an amount ranging from about 60 mg to 3200 mg;
(e) said tetrahydrobiopterin is in an amount ranging from about 25 mg to 3250 mg;
(f) said N-acetylcysteine is in an amount ranging from about 90 mg to 50 g; and
(g) said selenium is in an amount ranging from about 0.015 mg to 1 mg.

22. The natural composition in accordance with claim 9, wherein said high oxygen radical absorbance capacity blend comprises berry extracts, wherein said berry extracts comprise:
(a) strawberry extract;
(b) blueberry extract;
(c) blackberry extract;
(d) cranberry extract; and
(e) raspberry extract.

23. The natural composition in accordance with claim 22, wherein said high oxygen radical absorbance capacity blend is in an amount ranging from about 5 mg to 100 mg.

24. The natural composition in accordance with claim 10, wherein:
(a) said magnesium is in an amount ranging from about 15 mg to 1100 mg; and
(b) said calcium is in an amount ranging from about 5 mg to 750 mg.

25. The natural composition in accordance with claim 8, wherein said trace minerals further comprise:
(a) iodine;
(b) copper;
(c) manganese; and
(d) molybdenum.

26. The natural composition in accordance with claim 25, wherein:
(a) said iodine is in an amount ranging from about 25 mcg to 200 mcg;
(b) said copper is in an amount ranging from about 0.5 mg to about 4 mg;
(c) said manganese is in an amount ranging from about 0.5 mg to about 4 mg; and
(d) said molybdenum is in an amount ranging from about 25 mcg to 125 mcg.

27. The natural composition in accordance with claim 1, wherein said natural composition further comprises cell messenger agents.

28. The natural composition in accordance with claim 27, wherein said cell messenger agents comprise:
(a) inositol; and
(b) choline.

29. The natural composition in accordance with claim 28, wherein:
(a) said inositol is in an amount ranging from about 1 mg to 600 mg; and
(b) said choline is in an amount ranging from about 1 mg to 600 mg.

30. The natural composition in accordance with claim 28, wherein said cell messenger agents further comprise:
(a) boron;
(b) silica; and
(c) lutein.

31. The natural composition in accordance with claim 30, wherein:
(a) said boron is in an amount ranging from about 0.025 mg to about 50 mg;
(b) said silica is in an amount ranging from about 0.025 mg to about 100 mg; and
(c) said lutein is in an amount ranging from about 100 mcg to about 5 mg.

32. The natural composition in accordance with claim 1, wherein said natural composition further comprises one or more growth factors, wherein said one or more growth factors are adapted and formulated as an adjunct to said natural composition as a method to facilitate effective treatment of diseases, complications, conditions, or disorders including diabetes, inflammation, joint and muscle pain, muscle weakness, fatigue, chronic viral infections, cancer, nasal and sinus congestion, respiratory complications, digestive problems, memory loss, neuropathy, and psychological conditions including depression, mood swings, anger, anxiety, and confusion, and wherein said growth factors are further adapted and formulated to aid in reducing body fat and increasing or maintaining lean body tissue, thereby potentially leading to weight loss.

33. The natural composition in accordance with claim 32, wherein said adjunct is defined as a homeopathic formulation.

34. The natural composition in accordance with claim 33, wherein said homeopathic formulation comprises a molar concentration of between 1×10⁻2 and 1×10⁻1,250,000 of said one or more growth factors.

35. The natural composition in accordance with Clam 34, wherein said one or more growth factors are selected from the group consisting of:
(a) insulin-like growth factor I (IGF-I);
(b) insulin-like growth factor II (IGF-II);
(c) nerve growth factor (NGF);
(d) stem cell growth factor (SCF);
(e) fibroblast growth factor (FGF);
(f) granulocyte macrophage-colony stimulating growth factor (GM-CSF);
(g) granulocyte-colony stimulating growth factor (G-CSF);
(h) macrophage-colony stimulating growth factor (M-CSF);
(i) transforming growth factor α (TGFα);
() transforming growth factor β (TGβP);
(k) platelet-derived growth factor (PDGF);
(I) tumor necrosis factor α (TNFα); and
(m) tumor necrosis factor β (TNFβ).

36. A natural composition adapted to modulate multiple pathophysiological processes in order to facilitate homeostatic glucose metabolism as a method for the etiological prevention and treatment of diseases, complications, conditions, or disorders associated with chronic or long-term destabilization of glucose metabolism, and wherein said natural composition is further adapted to facilitate effective treatment of diseases, complications, conditions, or disorders including diabetes, inflammation, joint and muscle pain, muscle weakness, fatigue, chronic viral infections, cancer, nasal and sinus congestion, respiratory complications, digestive problems, memory loss, neuropathy, and psychological conditions including depression, mood swings, anger, anxiety, and confusion, said natural composition also adapted to aid in reducing body fat and increasing or maintaining lean body tissue, thereby potentially leading to weight loss, said natural composition comprising:
(a) vitamins;
(b) fatty acids;
(c) amino acids;
(d) phytochemicals;
(e) trace minerals;
(f) antioxidants;
(g) metals;
(h) cell messenger agents; and
(i) one or more growth factors.

37. A method for facilitating homeostatic glucose metabolism in a patient for the etiological treatment and etiological prevention of diseases, complications, conditions, or disorders associated with chronic or long-term destabilization of glucose metabolism, said method comprising:
(a) administering a patient an effective amount or dosage of a natural composition comprising vitamins, fatty acids, amino acids, phytochemicals, trace minerals, antioxidants, and metals.

38. The method in accordance with claim 37, wherein said natural composition further comprises:
(a) cell messenger agents; and
(b) growth factors.
